(19) United States
(12) Patent Application Publication
(10) Pub. No.: US 2007/0149580 A1
(43) Pub. Date: Jun. 28, 2007

(54) USE OF PEROXISOME PROLIFERATOR ACTIVATED RECEPTOR DELTA AGONISTS FOR THE TREATMENT OF MS AND OTHER DEMYELINATING DISEASES

(75) Inventors: Karen CHANDROSS, Somerset, NJ (US); Jean MERRILL, Whippany, NJ (US); Anne MINNICH, Flemington, NJ (US); Lan LEE, Pluckemin, NJ (US); Olga KHORKOVA, North Haledon, NJ (US); Yun LIU, Bridgewater, NJ (US)

Correspondence Address: ROSS J. OEHLER;SANOFI-AVENTIS U.S. LLC, 1041 ROUTE 202-206, MAIL CODE: D303A, BRIDGEWATER, NJ, 08807, US

(73) Assignee: AVENTIS PHARMACEUTICALS INC., Bridgewater, NJ (US)

(21) Appl. No.: 11/535,240

(22) Filed: Sep. 26, 2006

Related U.S. Application Data
(63) Continuation of application No. PCT/US05/10371, filed on Mar. 29, 2005.
(60) Provisional application No. 60/558,533, filed on Apr. 1, 2004.

(51) Int. Cl. A61K 31/426; A61K 31/192

(52) U.S. Cl. 514/365; 514/571

(57) ABSTRACT

A method for treating demyelinating diseases in a patient in need thereof by treatment with an effective amount of a PPAR delta agonist is disclosed. Demyelinating diseases that may be effectively treated by this method include but are not limited to multiple sclerosis, Charcot-Marie-Tooth disease, Pelizaeus-Merzbacher disease, encephalomyelitis, neuromyelitis optica, adrenoleukodystrophy, Guillian-Barre syndrome and disorders in which myelin forming glial cells are damaged including spinal cord injuries, neuropathies and nerve injury.

### CROSS-REFERENCE TO RELATED APPLICATIONS

This application is a continuation of International Application No. PCT/US2005/010371 that was filed on Mar. 29, 2005 which is incorporated herein by reference in its' entirety which also claims the benefit of priority of provisional U.S. Patent Application Ser. No. 60/558,533 filed on Apr. 1, 2004

### FIELD OF THE INVENTION

This invention relates to the use of PPAR delta agonists for the treatment of multiple sclerosis (MS) and other demyelinating diseases. This invention also relates to the use of certain compounds that are selective PPAR delta agonists for the treatment of MS and other demyelinating diseases.

### BACKGROUND OF THE INVENTION

The peroxisome proliferator-activated receptors (PPARs) comprise a subfamily of the nuclear receptor superfamily. Three closely related isoforms have been identified and cloned and are commonly known as PPAR alpha, PPAR gamma and PPAR delta. Each receptor subtype has a signature DNA binding domain (DBD) and a ligand-binding domain (LBD), both being necessary for ligand activated gene expression. PPARs bind as heterodimers with a retinoid X receptor. See J. Berger and D. E. Miller, Ann. Rev. Med., 2002, 53, 409-435.

PPAR delta (also known as PPAR beta) is expressed in a broad range of mammalian tissue, but little information regarding its biological functions or the full array of genes regulated by the receptor have been elucidated. However, it has recently been found that agonists may be useful to treat conditions such as dyslipedemia and certain dermatological conditions, while antagonists may be useful to treat osteoporosis or colorectal cancer (D. Sternbach, in Annual Reports in Medicinal Chemistry, Volume 38, A. M. Doherty, ed., Elsevier Academic Press, 2003 pp. 71-80).

PPAR delta appears to be significantly expressed in the CNS; however much of its function there still remains undiscovered. Of singular interest however, is the discovery that PPAR delta was expressed in rodent oligodendrocytes, the major lipid producing cells of the CNS (J. Granneman, et al., J. Neurosci. Res., 1998, 51, 563-573). Moreover, it was also found that a PPAR delta selective agonist was found to significantly increase oligodendroglial myelin gene expression and myelin sheath diameter in mouse cultures (I. Saluja et al., Glia, 2001, 33, 194-204).

Demyelinating conditions are manifested in the loss of myelin—the multiple dense layers of lipids and protein which cover many nerve fibers. These layers are provided by oligodendroglia in the central nervous system (CNS), and Schwann cells in the peripheral nervous system (PNS). In multiple sclerosis (MS), oligodendrocytes, the myelin forming cells in the CNS, are destroyed and axons are damaged, resulting in severely impaired neuronal activity and functional deficits, including palegia. In patients with demyelinating conditions, demyelination may be irreversible; it is usually accompanied or followed by axonal degeneration, and often by cellular degeneration. Demyelination can occur as a result of neuronal damage or damage to the myelin itself—whether due to aberrant immune responses, local injury, ischemia, metabolic disorders, toxic agents, or viral infections (Prineas and McDonald, Demyelinating Diseases. In Greenfield's Neuropathology, 6.sup.th ed. (Edward Arnold: New York, 1997) 813-811, Beers and Berkow, eds., The Merck Manual of Diagnosis and Therapy, 17.sup.th ed. (Whitehouse Station, N.J.: Merck Research Laboratories, 1999) 1299, 1437, 1473-76, 1483). However, newly formed oligodendrocyte progenitor cells are present throughout the areas of demyelination, suggesting the possibility of self-repair if these progenitor cells can be induced to undergo differentiation to mature oligodendrocytes.

Central demyelination (demyelination of the CNS) occurs in several conditions, often of uncertain etiology, that have come to be known as the primary demyelinating diseases. Of these, multiple sclerosis is the most prevalent. Other primary demyelinating diseases include adrenoleukodystrophy (ALD), adrenomyeloneuropathy, AIDS-vacuolar myelopathy, HTLV-associated myelopathy, Leber's hereditary optic atrophy, progressive multifocal leukoencephalopathy (PML), subacute sclerosing panencephalitis, and tropical spastic paraparesis. In addition, there are acute conditions in which demyelination can occur in the CNS, e.g., acute disseminated encephalomyelitis (ADEM) and acute viral encephalitis. Furthermore, acute transverse myelitis, a syndrome in which an acute spinal cord transection of unknown cause affects both gray and white matter in one or more adjacent thoracic segments, can also result in demyelination.

MS is a chronic, devastating, neurological disease that affects mostly young adults. The pathogenesis of MS is a complex process that leads to destruction of myelin and oligodendroglia, as well as axonal damage, in the brain and spinal cord (Prineas and McDonald, Demyelinating Diseases. In Greenfield's Neuropathology, 6.sup.th ed. (Edward Arnold: New York, 1997) 813-811, Trapp et al., N. Engl. J. Med., 338:278-85, 1998). Histopathologically, MS is characterized by inflammation, plaques of demyelination infiltrating cells in the CNS tissue, loss of oligodendroglia, and focal axonal injury (Prineas and McDonald, Demyelinating Diseases. In Greenfield's Neuropathology, 6.sup.th ed. (Edward Arnold: New York, 1997) 813-811). The disease is thought to result from aberrant immune responses to myelin, and possibly non-myelin, self-antigens (Bar-Or et al., J. Neuroimmiunol. 100:252-59, 1999, Hartung, H.-P., Current Opinion in Neurology, 8:191-99, 1995). Clinically, MS may follow a relapsing-remitting onset of occurrance, or it may take a chronically progressive course with increasing physical disability (Gold et al., Mol. Med. Today, 6:88-91, 2000). Typically, the symptoms of MS include lack of co-ordination, paresthesias, speech and visual disturbances, and weakness.

Current treatments for the various demyelinating conditions are often expensive, symptomatic, and only partially effective, and may cause undesirable secondary effects. Corticosteroids (oral prednisone at 60-100 mg/day, tapered over 2-3 weeks, or intravenous methylprednisolone at 500-1000 mg/day, for 3-5 days) represent the main form of therapy for MS. While these may shorten the symptomatic period during attacks, they may not affect eventual long-term disability. Long-term corticosteroid treatment is rarely justified, and can cause numerous medical complications, including osteoporosis, ulcers, and diabetes (Beers and Berkow, eds., The Merck Manual of Diagnosis and Therapy, 17. sup. th ed. (Whitehouse Station, N.J.: Merck Research Laboratories, 1999) 1299, 1437, 1473-76, 1483).

Immunomodulatory therapy with recombinant human interferon-.beta. (Betaseron and Avonex) and with co-polymer (Copaxon) slightly reduces the frequency of relapses in MS, and may help delay eventual disability (Beers and Berkow, eds., The Merck Manual of Diagnosis and Therapy, 17.sup.th ed. (Whitehouse Station, N.J.: Merck Research Laboratories, 1999) 1299, 1437, 1473-76, 1483). Both forms of interferon-.beta. and co-polymer are currently used as treatment modalities for MS, but all are exceedingly expensive. Immunosuppressive drugs (azathioprine, cladribine, cyclophosphamide, and methotrexate) are used for more severe progressive forms. However, they are not uniformly beneficial, and have significant toxic side effects. Several drugs (e.g., baclofen at 30-60 mg/day in divided doses) may reduce spasticity by inhibiting the spinal cord reflexes. Cautious and judicious use is required, though, because the drug-induced reduction in spasticity in MS patients often exacerbates weakness, thereby further incapacitating the patient.

Similarly, the current treatment for ALD, another devastating demyelinating disease, is relatively ineffective. Symptoms of ALD may include cortical blindness, corticospinal tract dysfunction, mental deterioration, and spasticity. Therapy to control the course of ALD may include bone marrow transplantation and dietary treatment (DiBiase et al., Ann. Ist. Super Sanita, 35:185-92, 1999), but inexorable neurological deterioration invariably occurs, ultimately leading to death [Krivit et al., Curr. Opin. Hematol., 6:377-82, 1999, (Beers and Berkow, eds., The Merck Manual of Diagnosis and Therapy, 17.sup.th ed. (Whitehouse Station, N.J.: Merck Research Laboratories, 1999) 1299, 1437, 1473-76, 1483). Some progress has been realized in the treatment of animals with EAE and EAN, by using glial cell transplants and growth factors, and by inhibiting adhesion molecules, autoantibodies, and cytokines (Njenga and Rodriguez, Current Opinion in Neurology, 9:159-64, 1996. However, none of these treatments has been shown to be beneficial in humans, and some require extensive neurosurgical intervention. Thus, it is clear from the foregoing that there exists a need for more effective, and less expensive and invasive methods to treat the varied array of demyelinating conditions, without producing the usually unavoidable and undesirable secondary side effects.

The present invention entails the use of a small molecule-activated regenerative approach to significantly augment current immunomodulatory therapies for the treatment of demyelinating disorders.

Compounds that are known to be selective PPAR delta are known in the art, in particular, compound of formula (1) generally known as GW 501516 described in WO 01/00603.

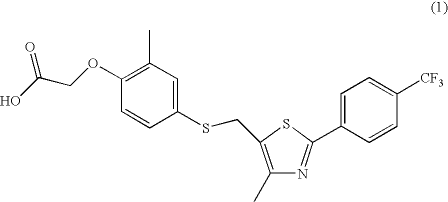

The compound of formula (2) also known as L165,041 has been disclosed in European Patent Application 28063 and in W097/28149 wherein it was identified as a selective PPAR delta agonist.

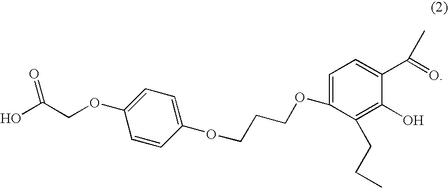

Due to the potential ability of Peroxisome Proliferator Activated Receptor Delta (PPAR delta) agonists to accelerate the differentiation of acutely isolated oligodendrocyte progenitor cells from rodent cerebrum and to significantly increase both myelin sheath diameter and myelin gene expression, there exists the potential for PPAR delta agonists to activate the PPAR delta pathway in oligodendrocyte progenitor cells and enhance neuronal repair by restoring the myelin sheath to demyelinated axons in demyelinating diseases, particularly MS.

### SUMMARY OF THE INVENTION

Thus in accordance with the practice of the present invention there is provided a method of treating a variety of demyelinating disease conditions with PPAR delta agonists, and in particular multiple sclerosis. In general, the disease conditions that can be treated in accordance with the practice of this invention include but not limited to multiple sclerosis, Charcot-Marie-Tooth disease, Pelizaeus-Merzbacher disease, encephalomyelitis, neuromyelitis optica, adrenoleukodystrophy, Guillian-Barrte syndrome and disorders in which myelin forming glial cells are damaged including spinal cord injuries, neuropathies and nerve injury. The diseases as disclosed herein can be treated by administering to a patient in need of such treatment a therapeutically effective amount of a PPAR delta agonist.

The present invention is also directed to the use of the compounds of formula (I) and formula (II) for the treatment of demyelinating diseases, and in particular multiple sclerosis.

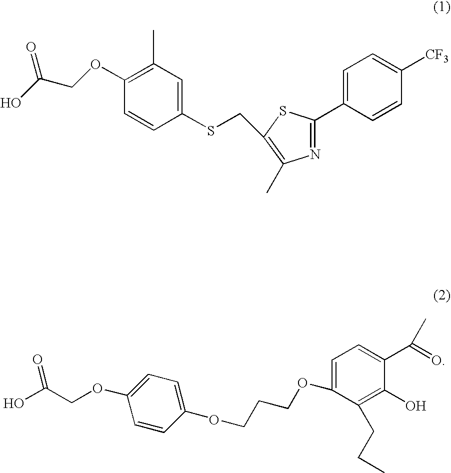

The present invention also comprises a method of treating multiple sclerosis in patients by administering a combination of the compounds of formula (1) or formula (2) or pharmaceutically acceptable salt thereof, with another compound known to be effective for the treatment of multiple sclerosis in therapeutically effective amounts. Compounds that are currently used to treat the disease are the disease-modifying agents such as the interferons (interferon beta 1-a, beta 1-b and alpha 2), glatiramer acetate or corticosteroids such as methylprednisolone and prednisone. Also, chemotherapeutic agents such as methotrexate, azathioprine, cladribine cyclophosphamide and cyclosporine.

### DETAILED DESCRIPTION OF THE INVENTION

As used herein, the expression “pharmaceutically acceptable carrier” means a non-toxic solvent, dispersant, excipient, adjuvant, or other material which is mixed with the compound of the present invention in order to permit the formation of a pharmaceutical composition, i.e., a dosage form capable of administration to the patient. One example of such a carrier is a pharmaceutically acceptable oil typically used for parenteral administration.

The term “pharmaceutically acceptable salts” as used herein means that the salts of the compounds of the present invention can be used in medicinal preparations. Other salts may, however, be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, hydrobromic acid, sulfuric acid, methanesulfonic acid, 2-hydroxyethanesulfonic acid, p-toluenesulfonic acid, fumaric acid, maleic acid, hydroxymaleic acid, malic acid, ascorbic acid, succinic acid, glutaric acid, acetic acid, salicylic acid, cinnamic acid, 2-phenoxybenzoic acid, hydroxybenzoic acid, phenylacetic acid, benzoic acid, oxalic acid, citric acid, tartaric acid, glycolic acid, lactic acid, pyruvic acid, malonic acid, carbonic acid or phosphoric acid. The acid metal salts such as sodium monohydrogen orthophosphate and potassium hydrogen sulfate can also be formed. Also, the salts so formed may present either as mono- or di- acid salts and can exist either as hydrated or can be substantially anhydrous. Furthermore, where the compounds of the invention carry an acidic moiety, suitable pharmaceutically acceptable salts thereof may include alkali metal salts, e.g. sodium or potassium salts; alkaline earth metal salts, e.g. calcium or magnesium salts; and salts formed with suitable organic ligands, e.g. quaternary ammonium salts.

The term “therapeutically effective amount” as used herein means an amount of the compound, which is effective in treating the named disorder or condition.

As used herein, the expression “pharmaceutically acceptable carrier” means a non-toxic solvent, dispersant, excipient, adjuvant, or other material which is mixed with the compound of the present invention in order to permit the formation of a pharmaceutical composition, i.e., a dosage form capable of administration to the patient. One example of such a carrier is a pharmaceutically acceptable oil typically used for parenteral administration.

The invention also provides pharmaceutical compositions comprising one or more of the compounds according to this invention in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. Alternatively, the compositions may be presented in a form suitable for once-weekly or once-monthly administration; for example, an insoluble salt of the active compound, such as the decanoate salt, may be adapted to provide a depot preparation for intramuscular injection. An erodible polymer containing the active ingredient may be envisaged. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. Flavored unit dosage forms contain from 1 to 100 mg, for example 1, 2, 5, 10, 25, 50 or 100 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

In the treatment of various disease states as described herein, a suitable dosage level is about 0.01 to 250 mg/kg per day, preferably about 0.05 to 100 mg/kg per day, and especially about 0.05 to 20 mg/kg per day. The compounds may be administered on a regimen of 1 to 4 times per day.

In one aspect of this invention there is disclosed a method for treating demyelinating diseases in a patient comprising administration of a therapeutically effective amount of a hPPAR delta agonist.

In a further aspect of this embodiment, the hPPAR delta agonist is a selective agonist.

In another aspect of this embodiment is disclosed a method wherein the demylenating disease is selected from the group consisting of multiple sclerosis, Charcot-Marie-Tooth disease, Pelizaeus-Merzbacher disease, encephalomyelitis, neuromyelitis optica, adrenoleukodystrophy, Guillian-Barre syndrome and disorders in which myelin forming glial cells are damaged including spinal cord injuries, neuropathies and nerve injury.

In a further aspect of this embodiment is disclosed the method wherein the demylenating disease is multiple sclerosis.

In yet another aspect of this embodiment is disclosed the method wherein the agonist is selected from group consisting of compound of formula (1) and formula (2)

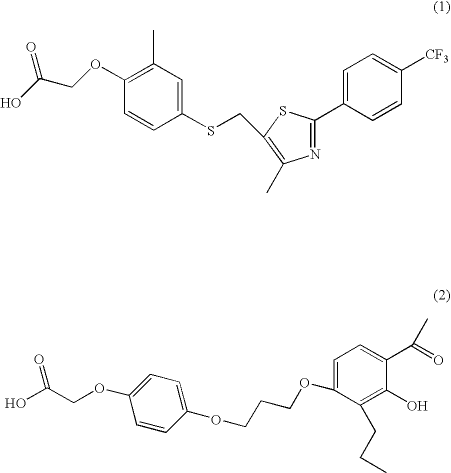

In another embodiment disclosed in the present invention is a pharmaceutical composition comprising a compound selected from the group consisting of compound of formula (1) and formula (2) in an amount effective for treating multiple sclerosis, Charcot-Marie-Tooth disease, Pelizaeus-Merzbacher disease, encephalomyelitis, neuromyelitis optica, adrenoleukodystrophy, Guillian-Barre syndrome and disorders in which myelin forming glial cells are damaged including spinal cord injuries, neuropathies and nerve injury in combination with at least on pharmaceutically acceptable carrier

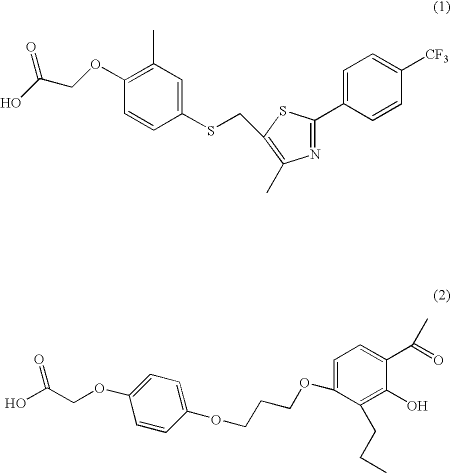

In a further aspect of this embodiment is disclosed a pharmaceutical composition comprising an amount effective in treating multiple sclerosis.

### BRIEF DESCRIPTION OF THE DRAWINGS

FIG. 1. Illustrates the enhancement of myelin basic protein (MBP) immunoreactivity in cultured rat oligodendrocytes by PPAR delta agonists.

FIG. 2. This graph shows the enhancement of MBP mRNA in cultured rat oligodendrocytes by compound 1.

FIG. 3. This graph shows the enhancement of MBP mRNA in cultured rat oligodendrocytes by compound 2.

FIG. 4A. Illustrates the effect of compound 1 on transcriptional markers that confirm PPAR delta agonist pathway activation in cultured rat oligodendrocytes.

FIG. 4B. Further illustrates the effect of compound 1 on transcriptional markers that confirm PPAR delta agonist pathway activation in cultured rat oligodendrocytes, showing that ADRP mRNA is upregulated in cultured rat oligodendrocytes.

FIG. 5. Shows the increase in the number of O4 immunopositive cells in mixed cultures of human oligodendrocytes effected by compound 1.

FIG. 6. Shows the increase in the number of O4 immunopositive cells in mixed cultures of human oligodendrocytes effected by compound 2.

### COMPOUND EXAMPLES

Compound of formula (1) (GW501516) can be prepared as published in WO 01/00603. Compound of formula (2) (L165,041) can be prepared as described in WO 97/28149.

### BIOLOGICAL EXAMPLES

The following test protocols are used to ascertain the biological properties of the compounds of this invention. The following examples are being presented to further illustrate the invention. However, they should not be construed as limiting the invention in any manner.

The PPAR delta agonists of the present invention are evaluated in in vitro and in vivo models for their ability to promote myelin expression and enhance regenerative processes.

The optimum nuclear receptor selectivity profile is determined by the GAL4/luciferase reporter assays. A rodent cellular assay shows the compound's ability to direct/accelerate differentiation of cultured oligodendrocyte progenitor cells to mature oligodendrocytes.

Specific biological assays suggesting efficacy for the treatment of MS are lysolecithin induced demyelination and experimental allergic encephalomyelitis performed in rodents.

Determination of EC50 Values of PPAR Agonists in the Cellular PPAR Delta Assay

Principle

The potency of substances, which bind to human PPAR delta and activate it in an agonistic manner, is analyzed using a stably transfected HEK cell line (HEK=human embryo kidney) which is referred to here as PPAR delta reporter cell line. The PPAR delta reporter cell line contains two genetic elements, a luciferase reporter element (pdeltaM-GAL4-Luc-Zeo) and a PPAR delta fusion protein (GR-GAL4-humanPPAR delta-LBD), which mediates expression of the luciferase reporter element depending on a PPAR delta ligand. The stably and constitutively expressed fusion protein GR-GAL4-humanPPAR delta-LBD binds in the cell nucleus of the PPAR delta reporter cell line via the GAL4 protein portion to the GAL4 DNA binding motifs 5′-upstream of the luciferase reporter element which is stably integrated in the genome of the cell line. There is only little expression of the luciferase reporter gene in the absence of a PPAR delta ligand if fatty acid-depleted fetal calf serum (cs-FCS) is used in the assay. PPAR delta ligands bind and activate the PPAR delta fusion protein and thereby stimulate expression of the luciferase reporter gene. The luciferase, which is formed can be detected by means of chemiluminescence via an appropriate substrate.

Construction of the PPAR Delta Reporter Cell Line:

The production of the stable PPAR delta reporter cell line is based on a stable HEK-cell clone which was stably transfected with a luciferase reporter element. This step was already described above in the section “construction of the PPAR alpha reporter cell line”. In a second step, the PPAR delta fusion protein (GR-GAL4-humanPPAR delta-LBD was stably introduced into this cell clone. For this purpose, the cDNA coding for the N-terminal 76 amino acids of the glucocorticoid receptor (Accession # P04150) was linked to the cDNA section coding for amino acids 1-147 of the yeast transcription factor GAL4 (Accession # P04386). The cDNA of the ligand-binding domain of the human PPAR delta receptor (amino acids S139-Y441; Accession # L07592) was cloned in at the 3′-end of this GR-GAL4 construct. The fusion construct prepared in this way (GR-GAL4-humanPPAR delta-LBD) was recloned into the plasmid pcDNA3 (Invitrogen) in order to enable constitutive expression by the cytomegalovirus promoter. This plasmid was linearized with a restriction endonuclease and stably transfected into the previously described cell clone containing the luciferase reporter element. The resulting PPAR delta reporter cell line which contains a luciferase reporter element and constitutively expresses the PPAR delta fusion protein (GR-GAL4-human PPAR delta-LBD) was isolated by selection with zeocin (0.5 mg/ml) and G418 (0.5 mg/ml).

Assay Procedure and Evaluation:

The activity of PPAR delta agonists is determined in a 3-day assay, which is described below:

Day 1

The PPAR delta reporter cell line is cultivated to 80% confluence in DMEM (# 41965-039, Invitrogen) which is mixed with the following additions: 10% cs-FCS (fetal calf serum; #SH-30068.03, Hyclone), 0.5 mg/ml zeocin (#R250-01, Invitrogen), 0.5 mg,/ml G418 (#10131-027, Invitrogen), 1% penicillin-streptomycin solution (#15140-122, Invitrogen) and 2 mM L-glutamine (#25030-024, Invitrogen). The cultivation takes place in standard cell culture bottles (# 353112, Becton Dickinson) in a cell culture incubator at 37° C. in the presence of 5% CO₂. The 80%-confluent cells are washed once with 15 ml of PBS (#14190-094, Invitrogen), treated with 3 ml of trypsin solution (#25300-054, Invitrogen) at 37° C. for 2 min, taken up in 5 ml of the DMEM described and counted in a cell counter. After dilution to 500.000 cells/ml, 35,000 cells are seeded in each well of a 96 well microtiter plate with a clear plastic base (#3610, Corning Costar). The plates are incubated in the cell culture incubator at 37° C. and 5% CO₂ for 24 h.

Day 2

PPAR delta agonists to be tested are dissolved in DMSO in a concentration of 10 mM. This stock solution is diluted in DMEM (#41965-039, Invitrogen) which is mixed with 5% cs-FCS (#SH-30068.03, Hyclone), 2 mM L-glutamine (#25030-024, Invitrogen) and the previously described antibiotics (zeocin, G418, penicillin and streptomycin). Test substances are tested in 11 different concentrations in the range from 10 μM to 100 pM. More potent compounds are tested in concentration ranges from 1 μM to 10 pM or between 100 nM and 1 pM.

The medium of the PPAR delta reporter cell line seeded on day 1 is completely removed by aspiration, and the test substances diluted in medium are immediately added to the cells. The dilution and addition of the substances is carried out by a robot (Beckman FX). The final volume of the test substances diluted in medium is 100 μl per well of a 96 well microtiter plate. The DMSO concentration in the assay is less than 0.1% v/v in order to avoid cytotoxic effects of the solvent. Each plate was charged with a standard PPAR delta agonist, which was likewise diluted in 11 different concentrations, in order to demonstrate the functioning of the assay in each individual plate. The assay plates are incubated in an incubator at 37° C. and 5% CO₂ for 24 h.

Day 3

The PPAR delta reporter cells treated with the test substances are removed from the incubator, and the medium is aspirated off. The cells are lyzed by pipetting 50 μl of Bright Glo reagent (from Promega) into each well of a 96 well microtiter plate. After incubation at room temperature in the dark for 10 minutes, the microtiter plates are measured in the luminometer (Trilux from Wallac). The measuring time for each well of a microtiter plate is 1 sec.

Evaluation:

The raw data from the luminometer are transferred into a Microsoft Excel file. Dose-effect plots and EC50 values of PPAR agonists are calculated using the XL.Fit program as specified by the manufacturer (IDBS).

Determination of EC₅₀ Values of PPAR Agonists in the Cellular PPAR Alpha Assay:

Principle

The potency of substances which bind to human PPAR alpha and activate it in an agonistic manner is analyzed using a stably transfected HEK cell line (HEK=human embryo kidney) which is referred to here as PPAR alpha reporter cell line. It contains two genetic elements, a luciferase reporter element (pdeltaM-GAL4-Luc-Zeo) and a PPAR alpha fusion protein (GR-GAL4-humanPPAR alpha-LBD) which mediates expression of the luciferase reporter element depending on a PPAR alpha ligand. The stably and constitutively expressed fusion protein GR-GAL4-humanPPAR alpha-LBD binds in the cell nucleus of the PPAR alpha reporter cell line via the GAL4 protein portion to the GAL4 DNA binding motifs 5′-upstream of the luciferase reporter element which is stably integrated in the genome of the cell line. There is only weak expression of the luciferase reporter gene in the absence of a PPAR alpha ligand if fatty acid-depleted fetal calf serum (cs-FCS) is used in the assay. PPAR alpha ligands bind and activate the PPAR alpha fusion protein and thereby stimulate the expression of the luciferase reporter gene. The luciferase which is formed can be detected by means of chemiluminescence via an appropriate substrate.

Construction of the PPAR Alpha Reporter Cell Line:

The PPAR alpha reporter cell line was prepared in two stages. Firstly, the luciferase reporter element was constructed and stably transfected into HEK cells. For this purpose, five binding sites of the yeast transcription factor GAL4 (Accession # AF264724) were cloned in 5′-upstream of a 68 bp-long minimal MMTV promoter (Accession # V01175). The minimal MMTV promoter section contains a CCAAT box and a TATA element in order to enable efficient transcription by RNA polymerase II. The cloning and sequencing of the GAL4-MMTV construct took place in analogy to the description of Sambrook J. et. al. (Molecular cloning, Cold Spring Harbor Laboratory Press, 1989). Then the complete Photinus pyralis gene (Accession # M15077) was cloned in 3′-downstream of the GAL4-MMTV element. After sequencing, the luciferase reporter element consisting of five GAL4 binding sites, MMTV promoter and luciferase gene was recloned into a plasmid which confers zeocin resistance in order to obtain the plasmid pdeltaM-GAL4-Luc-Zeo. This vector was transfected into HEK cells in accordance with the statements in Ausubel, F. M. et al. (Current protocols in molecular biology, Vol. 1-3, John Wiley & Sons, Inc., 1995). Then zeocin-containing medium (0.5 mg/ml) was used to select a suitable stable cell clone which showed very low basal expression of the luceriferase gene.

In a second step, the PPAR alpha fusion protein (GR-GAL4-humanPPAR alpha-LBD was introduced into the stable cell clone described. For this purpose, initially the cDNA coding for the N-terminal 76 amino acids of the glucocorticoid receptor (Accession # P04150) was linked to the cDNA section coding for amino acids 1-147 of the yeast transcription factor GAL4 (Accession # P04386). The cDNA of the ligand-binding domain of the human PPAR alpha receptor (amino acids S167-Y468; Accession # S74349) was cloned in at the 3′-end of this GR-GAL4 construct. The fusion construct prepared in this way (GR-GAL4-humanPPAR alpha-LBD) was recloned into the plasmid pcDNA3 (Invitrogen) in order to enable constitutive expression therein by the cytomegalovirus promoter. This plasmid was linearized with a restriction endonuclease and stably transfected into the previously described cell clone containing the luciferase reporter element. The finished PPAR alpha reporter cell line which contains a luciferase reporter element and constitutively expresses the PPAR alpha fusion protein (GR-GAL4-human PPAR alpha-LBD) was isolated by selection with zeocin (0.5 mg/ml) and G418 (0.5 mg/ml).

Assay Procedure:

The activity of PPAR alpha agonists is determined in a 3-day assay, which is described below:

Day 1

The PPAR alpha reporter cell line is cultivated to 80% confluence in DMEM (# 41965-039, Invitrogen) which is mixed with the following additions: 10% cs-FCS (fetal calf serum; #SH-30068.03, Hyclone), 0.5 mg/ml zeocin (#R250-01, Invitrogen), 0.5 mg/ml G418 (#10131-027, Invitrogen), 1% penicillin-streptomycin solution (#15140-122, Invitrogen) and 2 mM L-glutamine (#25030-024, Invitrogen). The cultivation takes place in standard cell culture bottles (# 353112, Becton Dickinson) in a cell culture incubator at 37° C. in the presence of 5% CO₂. The 80%-confluent cells are washed once with 15 ml of PBS (#14190-094, Invitrogen), treated with 3 ml of trypsin solution (#25300-054, Invitrogen) at 37° C. for 2 min, taken up in 5 ml of the DMEM described and counted in a cell counter. After dilution to 500.000 cells/ml, 35,000 cells are seeded in each well of a 96 well microtiter plate with a clear plastic base (#3610, Corning Costar). The plates are incubated in the cell culture incubator at 37° C. and 5% CO₂ for 24 h.

Day 2

PPAR alpha agonists to be tested are dissolved in DMSO in a concentration of 10 mM. This stock solution is diluted in DMEM (#41965-039, Invitrogen) which is mixed with 5% cs-FCS (#SH-30068.03, Hyclone), 2 mM L-glutamine (#25030-024, Invitrogen) and the previously described antibiotics (zeocin, G418, penicillin and streptomycin). Test substances are tested in 11 different concentrations in the range from 10 μM to 100 pM. More potent compounds are tested in concentration ranges from 1 μM to 10 pM or between 100 nM and 1 pM.

The medium of the PPAR alpha reporter cell line seeded on day 1 is completely removed by aspiration, and the test substances diluted in medium are immediately added to the cells. The dilution and addition of the substances is carried out by a robot (Beckman FX). The final volume of the test substances diluted in medium is 100 μl per well of a 96 well microtiter plate. The DMSO concentration in the assay is less than 0.1% v/v in order to avoid cytotoxic effects of the solvent. Each plate was charged with a standard PPAR alpha agonist, which was likewise diluted in 11 different concentrations, in order to demonstrate the functioning of the assay in each individual plate. The assay plates are incubated in an incubator at 37° C. and 5% CO₂ for 24 h.

Day 3

The PPAR alpha reporter cells treated with the test substances are removed from the incubator, and the medium is aspirated off. The cells are lyzed by pipetting 50 μl of Bright Glo reagent (from Promega) into each well of a 96 well microtiter plate. After incubation at room temperature in the dark for 10 minutes, the microtiter plates are measured in the luminometer (Trilux from Wallac). The measuring time for each well of a microtiter plate is 1 sec.

Evaluation:

The raw data from the luminometer are transferred into a Microsoft Excel file. Dose-effect plots and EC50 values of PPAR agonists are calculated using the XL.Fit program as specified by the manufacturer (IDBS).

Determination of EC50 Values of PPAR Agonists in the Cellular PPAR Gamma Assay

Cell Based PPAR Gamma Assay Protocol

To perform cell based assays a luciferase assay is performed in 96 well plates as follows:

Day 1: Plating of Cells:

Wash cells grown to 80-90% confluency once in PBS
Trypsinize for 2 min
Add 15 ml assay medium (DMEM, Invitrogen, Cat. No. 41965-039; 5% Charcoal/Dextran Treated FBS, Hyclone, Cat. No. SH30068; 0.5 mg/ml Zeocin, Invitrogen, Cat. No. 46-0072; 0.5 mg/ml Geneticin, Invitrogen, Cat. No. 10131-027; 1% Penicillin/Streptomycin, Invitrogen, Cat. No. 15140-122; 2 mM L-Glutamine, Invitrogen, Cat. No. 25030-024; 7.5 μg/ml Blasticidin S HCl, Invitrogen, Cat. No. R210-01; 1 μg/ml Doxycycline, Clontech, Cat. No. 8634-1)
Count cells
Dilute cells in assay medium to 500.000 cells/ml
Dispense 100 μl of cell suspension per well in clear bottom Corning plates (makes 50.000 cells/well)
Incubate for 24 h at 37° C., 5% CO2
   Day 2: Dosing with Test Compounds:
Solve test compounds in DMSO to make a 10 mM stock solution
Dilute compound to appropriate concentration in assay medium (DMEM, Invitrogen, Cat. No. 41965-039; 5% Charcoal/Dextran Treated FBS, Hyclone, Cat. No. SH30068; 0.5 mg/ml Zeocin, Invitrogen, Cat. No. 46-0072; 0.5 mg/ml Geneticin, Invitrogen, Cat. No. 10131-027; 1% Penicillin/Streptomycin, Invitrogen, Cat. No. 15140-122; 2 mM L-Glutamin, Invitrogen, Cat. No. 25030-024; 7.5 μg/ml Blasticidin S HCl, Invitrogen, Cat. No. R210-01; 1 μg/ml Doxycycline, Clontech, Cat. No. 8634-1) (regular FCS is harboring\free fatty acids interfering with the PPAR ligand binding domains).
Aspirate medium (cells are quite sensitive at this step; make sure that cells are no longer than 1 min without being covered by medium)
Transfer diluted compounds to 96 wells (100 μl medium including compound)
Make controls with standard compound (e.g. Rosiglitazon) as well as a DMSO control (0.1% DMSO)
Incubate cells for 24 h at 37° C. at 5% CO2

Dilution steps and addition of diluted compounds is done using a Beckman Biomek 2000 or Beckman FX robot.

Day 3: Cell Lysis and Measurement of Luciferase Activity:

Aspirate medium from cells
Freeze plates at −20° C. (optional)
Thaw plates for 30 min (if necessary)
Add 50 μl Bright-Glo-Luciferase Assay Reagent (Promega, Cat. No. E2650)
Incubate for 10 min in the dark
Measure luminescence 2 sec per well (Wallac Microbeta)
   Data Analysis:

Determination of EC50 values is done with Microsoft Exel in combination with XLFit (develop by IDBS) using the fitting algorithm #205.

Determination of EC50 Values in the Cellular Human RXR Receptor Assay

Cell Based RXR Assay Protocol

To perform cell based assays a luciferase assay is performed in 96 well plates as follows:

Day 1: Plating of Cells

Wash cells grown to 80-90% confluency once in PBS
Trypsinize for 2 min
Add 15 ml culture medium (DMEM, Invitrogen, Cat. No. 41965-039; 10% Charcoal/Dextran Treated FBS, Hyclone, Cat. No. SH30068; 0.5 mg/ml Zeocin, Invitrogen, Cat. No. 46-0072; 0.5 mg/ml Geneticin, Invitrogen, Cat. No. 10131-027; 1% Penicillin/Streptomycin, Invitrogen, Cat. No. 15140-122; 2 mM L-Glutamin, Invitrogen, Cat. No. 25030-024)
Count cells
Dilute cells in culture medium to 175.000 cells/ml
Dispense 200 μl of cell suspension per well in clear bottom Corning plates (makes 35.000 cells/well)
Incubate for 24 h at 37° C., 5% CO2
   Day 2: Dosing with Test Compounds
Solve test compounds in DMSO to make a 10 mM stock solution
Dilute compound to appropriate concentration in assay medium (DMEM w/o phenol-red, Invitrogen, Cat. No. 21063-029; 5% Charcoal/Dextran Treated FBS, Hyclone, Cat. No. SH30068; 0.5 mg/ml Zeocin, Invitrogen, Cat. No. 46-0072; 0.5 mg/ml Geneticin, Invitrogen, Cat. No. 10131-027; 1% Penicillin/Streptomycin, Invitrogen, Cat. No. 15140-122; 2 mM L-Glutamin, Invitrogen, Cat. No. 25030-024) (regular FCS is harboring free fatty acids interfering with the PPAR ligand binding domains).
Aspirate medium (cells are quite sensitive at this step; make sure that cells are no longer than 1 min without being covered by medium)
Transfer diluted compounds to 96 wells (100 μl medium including compound)
Make controls with standard compound (e.g. RPR258134) as well as a DMSO control (0.1% DMSO)
Incubate cells for 24 h at 37° C. at 5% CO2

Dilution steps and addition of diluted compounds is done using a Beckman Biomek 2000 or Beckman FX robot.

Day 3: Cell Lysis and Measurement of Luciferase Activity

Aspirate medium from cells
Freeze plates at −20° C. (optional)
Thaw plates for 30 min (if necessary)
Add 50 μl Bright-Glo-Luciferase Assay Reagent (Promega, Cat. No. E2650)
Incubate for 10 min in the dark
Measure luminescence 2 sec per well (Wallac Microbeta)
   Data Analysis

Determination of EC50 values is done with Microsoft Exel in combination with XLFit (develop by IDBS) using the fitting algorithm #205.

Table 1 shows the results if the reporter assays. The results show that compounds 1 and 2 are selective PPAR delta activators with low PPAR alpha, gamma and RXR activity.

TABLE 1
Reporter Assays

| | Human | Mouse | Human PPAR | Human PPAR | Human PPAR |
|---|---|---|---|---|---|
| | PPAR delta | PPAR delta | alpha | gamma | RXR¹ |
| Compound | EC₅₀ (μM) | EC₅₀ (μM) | EC₅₀ (μM) | EC₅₀ (μM) | EC₅₀ (μM) |
| 1 | <0.00457 | 7.8 (4x)*, | 1.3 | 3.97 (3.1x)* | No increase |
| | (29x)* | 6 (2x)*, | | | |
| | | 4 (2x)* | | | |
| 2 | 0.039 | 1 (2x)* | 1.1 | 1.1 (4.3x)* | No increase |
| | (27x)* | | | | |

*Value represents the fold increase over baseline luciferase activity.
¹Retinoid X receptor

### RAT/MICE Oligodendrocyte Cultures

Preparation of Cells:

1. Primary rat oligodendrocyte progenitor cells are obtained from the neocortex of newborn (postnatal days 2-3) rats or mice and are enriched, after removal of microglia, by mechanical separation from the astrocytic monolayer using a modification of the technique originally described by McCarthy and de Vellis (1980).
2. Remove the meninges from neonatal rat brain and mechanically dissociate tissue. Plate cells on T75 flasks and feed cells with DMEM/F12+10% FBS.
3. Collect oligodendrocytes growing on the astrocyte bed layer by shaking-off method fourteen days after the original prep date. Centrifuge the suspension and resuspend the cell pellet in serum free media (SFM; DMEM combined with 25 μg/ml transferring, 30 nM triiodothyronine, 20 nM hydrocortisone, 20 nM progesterone, 10 nM biotin, 1× trace elements, 30 nM selenium, 1 μg/ml putrescine, 0.1% BSA, 5 U/ml PenStrep, 10 μg/ml insulin) supplemented with the following growth factors: Platelet derived growth factor-AA (PDGF) and fibroblast growth factor-2 (FGF).
4. Plate the cells on PDL-coated dishes and incubate at 37° C. with 6-7% CO2.
5. Media components are replaced every 48 hr to keep the cells in a progenitor state.
   Progenitor Cell Passaging to Increase Cell Numbers for Screening Assays:
1. When the culture are confluent, rinse the culture with PBS, add trypsin and incubate for ˜2-3 min at 37° C.
2. Neutralize and centrifuge the cell suspension at 900 g for 5 min.
3. Resuspend the cell pellet in SFM+PDGF/FGF.
4. Feed the cells with fresh growth factors every 48 hrs to keep enrich for rapidly dividing progenitor cells.
5. Cells are passaged no more than 4-5 times prior to experimental assays.
6. All experiments involving oligodendrocyte progenitor cells were done using cells that were continuously maintained under these conditions. Greater than 95% of all cells were A2B5 immunopositive and expressed 2′3′-cyclic nucleotide 3′-phosphodiesterase II mRNA.
7. To generate mature oligodendrocytes, 24 h after plating progenitor cells were switched to SFM supplemented with or without IGF-I and grown under these conditions for 7 d prior to experimental assays.
8. Alternatively, the enriched rat Central Glia-4 (CG4) progenitor cell line may be used, which is maintained in base media (DMEM, with 2 mM glutamine, 1 mM sodium pyruvate, biotin (40 nM), insulin (1 μM) and N1) supplemented with 30% conditioned media from the B-104 neuroblastoma cell line. To induce differentiation, CG4 cells are switched to base media with 1% fetal calf serum (removed after 2 days) and insulin (500 nM). A2B5 and MBP immunoreactivity is used to confirm >95% enrichment in immature and mature cultures, respectively.
   Rat/Mouse Culture Compound Treatment:
1. Put 10,000-15,000 cells/well in 24-well PDL coated plates and culture the cells in presence of mitogen (10 ng/ml) overnight.
2. In the presence of mitogen:
   a. Next day, remove the old medium and add compounds in fresh medium (with mitogen)
   b. Compound dose response evaluations are performed at 6 different concentrations (10 □M, 1 □M, 100 nM, 10 nM, 1 nM, and 0.1 nM);
   c. Triplicates wells are run for each compound concentration.
3. In the absence of mitogen:
   a. Next day, remove the old medium and add compounds in fresh medium (without mitogen)
   b. Compound dose response evaluations are performed at 6 concentrations (10 □M, 1 □M, 100 nM, 10 nM, 1 nM, and 0.1 nM);
   c. Triplicates wells are run for each compound concentration.
4. Culture the treated cells for 7 d prior to using in experimental assays.

### Human Oligodendrocyte Cultures

Preparation of Cells:

1. Human neurospheres collected from E19.5-E22 human embryo cortex) are cultured for 2 weeks in progenitor media: DMEM/F12 containing 100 μg/ml transferring, 30 nM triiodothyronine, 20 nM hydrocortisone, 20 nM progesterone, 10 nM biotin, 1× trace elements, 30 nM selenium, 60 uM putrescine, 0.1% BSA, 5 U/ml PenStrep, 25 μg/ml insulin) supplemented with PDGF and FGF.
2. Neurospheres are dissociated with 20 U/ml papain at 37° C. for 30-50 min.
3. Cells are plated onto PDL coated dishes at density of 50,000-100,000 cell/well in progenitor media containing PDGF/FGF and incubated at 37° C. with 5-6% CO2.
4. Media and growth factors are replenished every 48 hr.
   Human Culture Compound Treatment:
1. 24 to 48 hr after plating remove the old medium and add compounds in fresh medium (with mitogen)
2. Compound dose response evaluations are performed at 3-6 different concentrations (10 □M, 1 □M, 100 nM, 10 nM, 1 nM, and 0.1 nM)
3. Triplicates wells are run for each compound concentration.
5. Culture the treated cells for 7 d prior to using in experimental assays.

Rat/Mouse/Human Oligodendrocyte Specific Immunostaining:

Following compound exposure, oligodendrocyte-specific antibodies are used to assess ability of compound to accelerate/promote oligodendrocyte differentiation (for example, O4, O1, or myelin basic protein immunoreactivity is over time between compound treated and untreated cultures).
1. Cells are plated onto poly-D-lysine treated 4-well chamber slides at 5×10³ to 20×10³ cells/well and grown as described above. Sequential staining is performed on oligodendrocyte populations with increasing degrees of cellular differentiation, as determined by days in vitro without PDGF and FGF.
2. Live staining for 30 min at 37° C. is used to detect oligodendrocyte stage specific cell surface marker expression (including A2B5, O4, and O1).
3. Subsequently, cells are fixed with 4% paraformaldehyde, 10 min, room temperature.
4. Fixed staining procedures are used to detect oligodendrocyte stage specific marker expression (including myelin basic protein, MBP).
5. Rinse with PBS.
6. Permeabilize with 0.1% Triton/0.01% NaAz diluted in 1×PBS for 10 min, room temperature.
7. Block with 5-10% goat serum in antibody dilution buffer (0.1% Triton-X 100 and 1% IgG-free bovine serum albumin; also used to dilute antibodies), 15 min, room temperature.
8. Add primary antibody diluted in antibody dilution buffer.
9. Incubate overnight, gently rocking, 40° C.
10. Next day, rinse with PBS 1×5 min, followed by 3×15 min each, room temperature.
11. Incubate with appropriate secondary antibodies, 45 min, room temperature.
12. Cell nuclei are stained with 4,6-diamidino-2-phenylindole (DAPI), 15 min, room temperature.
13. Rinse several times with PBS and evaluate using fluorescent microscopy.
14. The following conditions are compared over time and at different compound doses: PDGF/FGF alone, SFM alone, SFM-IGF1 alone, PDGF/FGF and compound, SFM and compound.
   Rat/Mouse/Human Bromodeoxyuridine (BrdU) Immunostaining:
   To Confirm that Compounds do not Promote Cell Proliferation.
1. Oligodendrocyte progenitor cells are labeled with 10 □M BrdU for 20 hr and then fixed with either 70% ethanol or 4% paraformaldehyde.
2. The cells are incubated successively with biotinylated mouse anti-BrdU and Streptavidin-Peroxidase, with three intervening washes with PBS.
3. Colormetric visualization of the BrdU immunoreactivity is developed with DAB and total cell numbers are assessed using the counter-stain hematoxylin.
4. BrdU immunopositive cells are counted by two independent observers.
   Rat/Mouse/Human Culture Image Analysis: Fluorescent microscopy is used to quantitate the extent of oligodendrocyte differentiation after compound exposure. This assay demonstrates that selective agonists accelerate/promote oligodendrocytes differentiation.
1. Manual Cell Counting: Four fields are randomly selected for each experimental condition and 500-600 cells are counted in each field. The percentage of MBP (or O4) immunpositive cells (mature process bearing cells with or without myelin sheets) versus DAPI positive cells (total cell number) cells are compared in the control and drug-treated groups.
2. Automated Cell Counting: Fluorescent microscopy was used to quantitate the extent of oligodendrocyte differentiation after compound exposure. Six fields/well are randomly selected to assess the number of differentiating oligodendrocytes among the total population (˜8 to 15×10³ cells are counted/well). Immunofluorescence images are obtained using a Zeiss AxioVision digital imaging system, with a Zeiss AxioCam HRc cooled CCD camera connected to the same microscope. All microscopic imaging parameters are set for acquiring images for the analysis of cellular immunofluorescence intensity. The percentage of MBP positive (differentiated) cells versus total cells (DAPI nuclear stained) is compared in the control versus drug-treated groups. Cellular autofluorescence was undetectable under the imaging conditions.
a) 3. Human oligodendrocyte differentiation assay: manually count total number of O4 immunopositive cells/well (bipolar and multipolar).

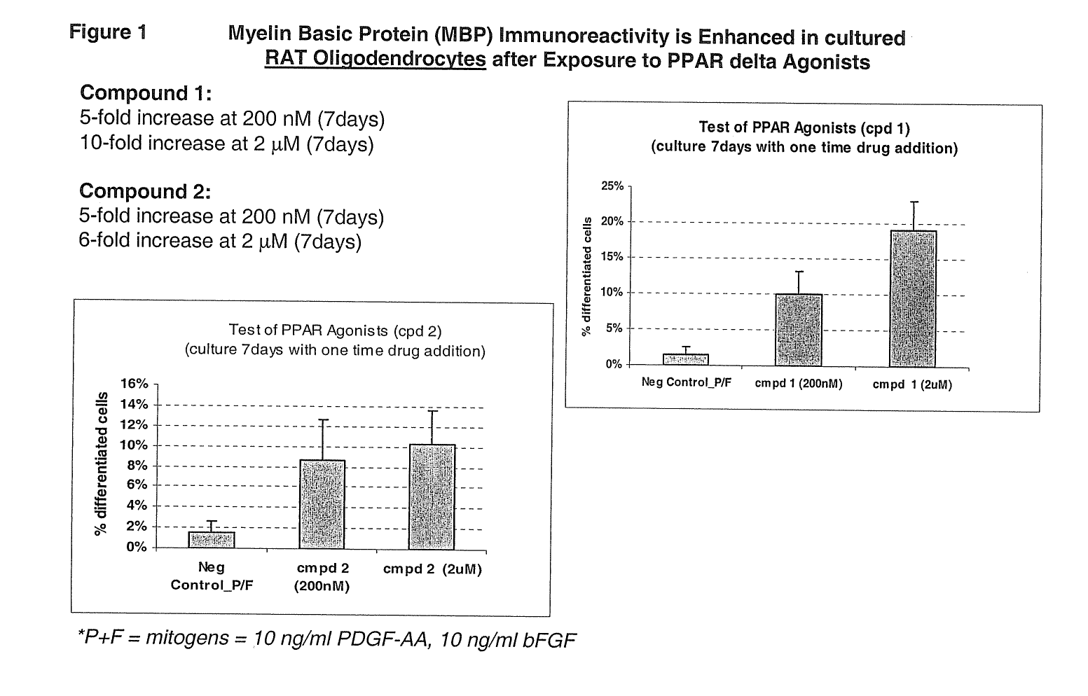

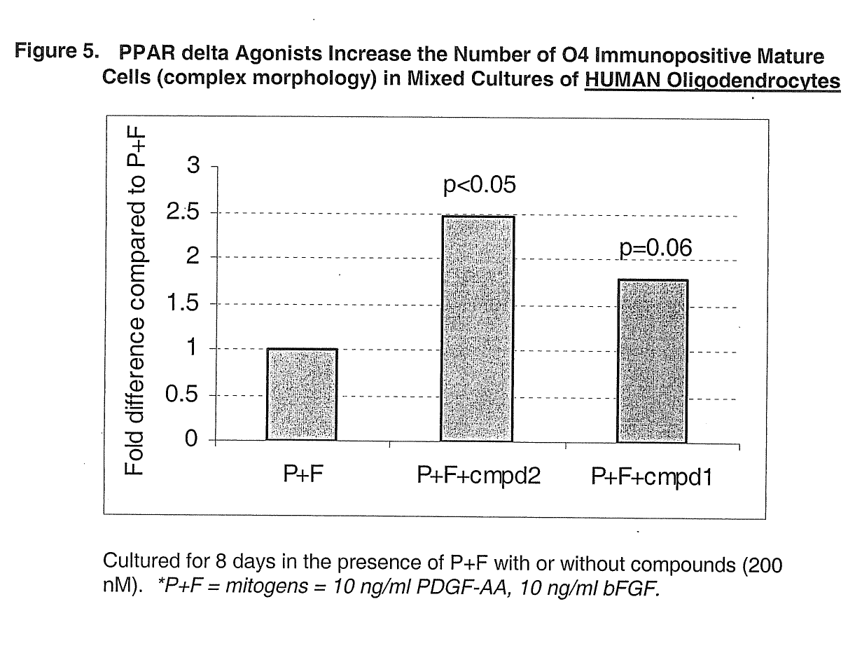

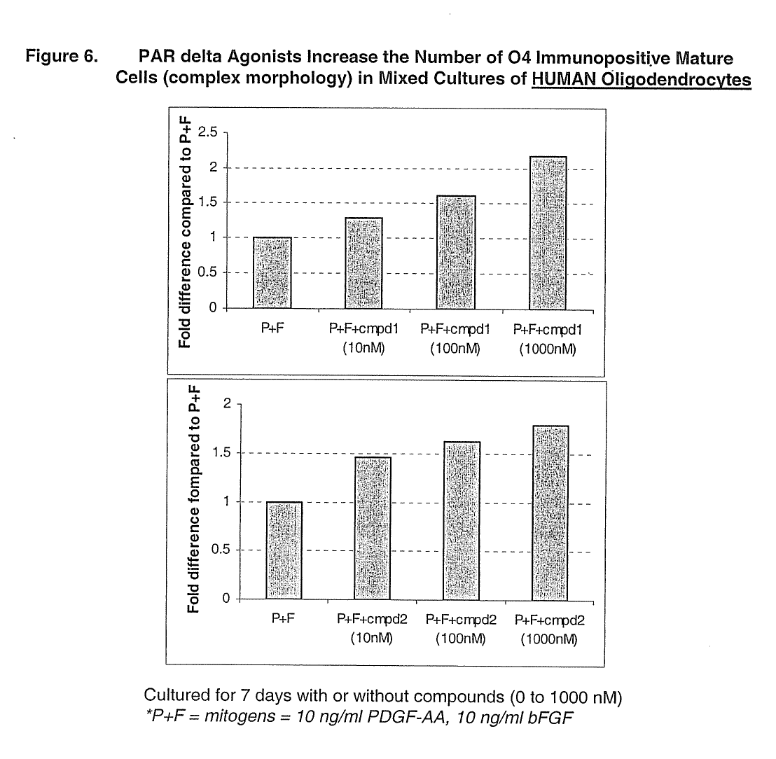

The results using rat oligodendrocyte cultures are shown in FIG. 1 and the results using human oligodendrocyte mixed cultures are shown in FIGS. 5 and 6. As the results show, PPAR delta agonists enhance or accelerate rat and human oligodendrocyte differentiation, as measured by increased myelin basic protein expression compared to untreated controls. This novel finding would suggest that compound 1 and compound 2 and selective PPAR delta agonists in general would be enhance, accelerate, or stimulate oligodendrocyte differentiation and myelin formation in vivo, in the diseased or injured CNS, including MS and other demyelinating disorders.

Rat/Mouse/Human Quantitative Polymerase Chain Reaction (PCR): To evaluate compound induced PPAR delta pathway activation and the extent of oligodendrocyte maturation (changes in mRNA levels).

1. Total RNA is extracted from cultured oligodendrocytes using TriZol reagent.
2. Subsequently, mRNA is treated with RNase-free DNase, repurified, and then converted to cDNA template using a RT reaction (Clontech Advantage RT for PCR Kit).
3. PPAR delta pathway member transcript expression is quantitated using Sybr Green PCR Master Mix.
4. The 18S ribosomal RNA primer/probe mix (186 bp product), suspended in Taqman 2×PCR Master Mix is used as an internal control.
5. Quantitative PCR is carried out using real-time Taqman™ technology (Gibson, et al., 1996) with a model 7700 Sequence Detector System (Applied Biosystems, Foster City, Calif.).
6. The results are analyzed using Sequence Detection Systems software version 1.91.

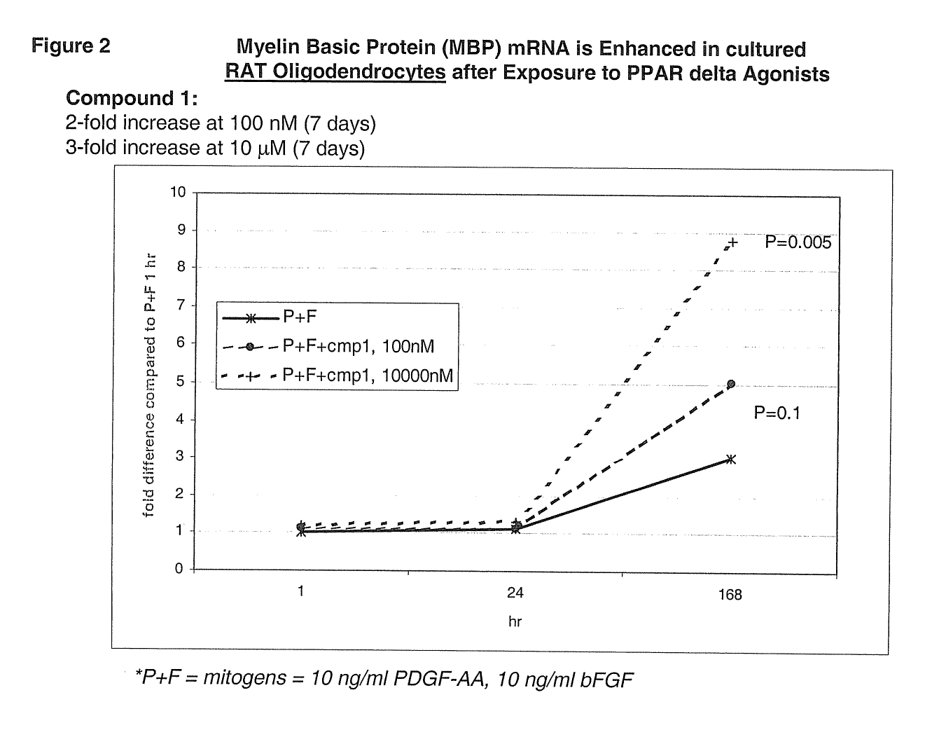

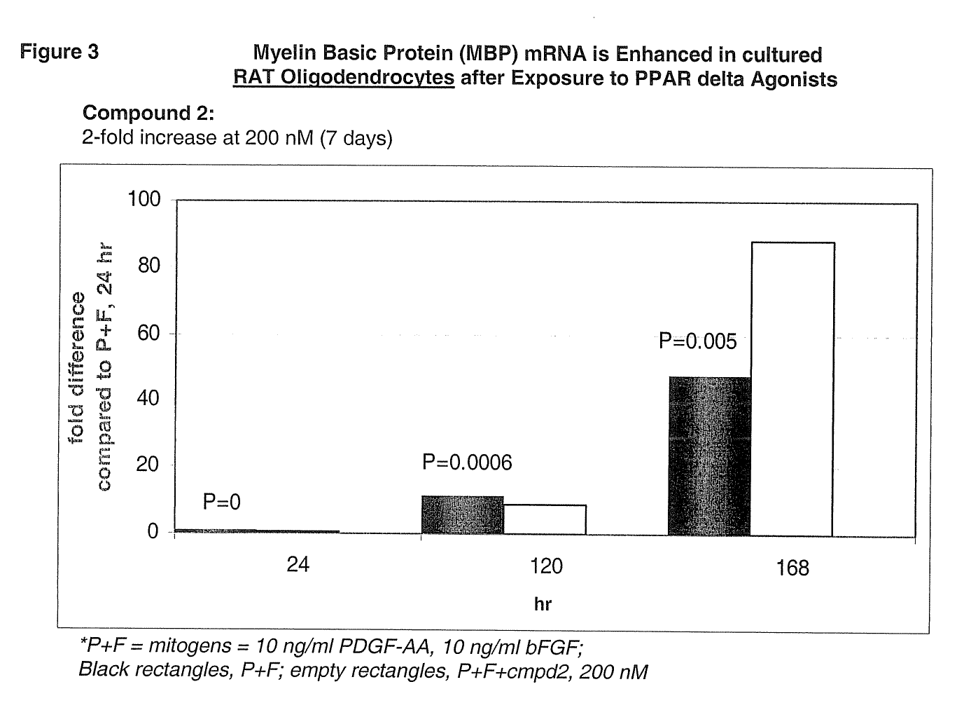

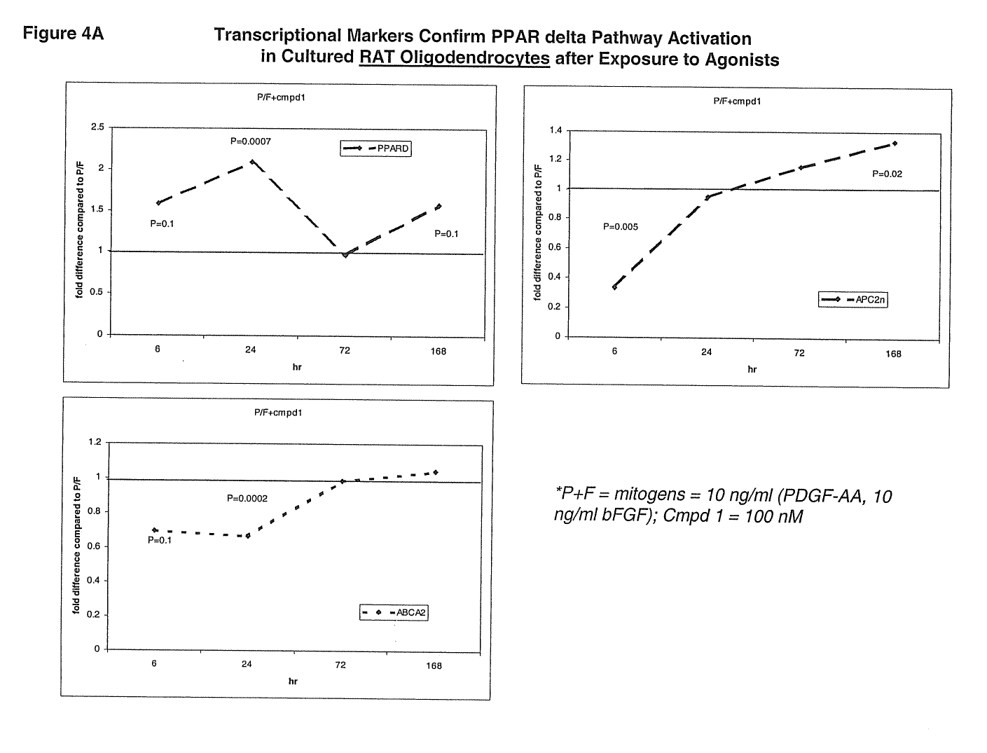

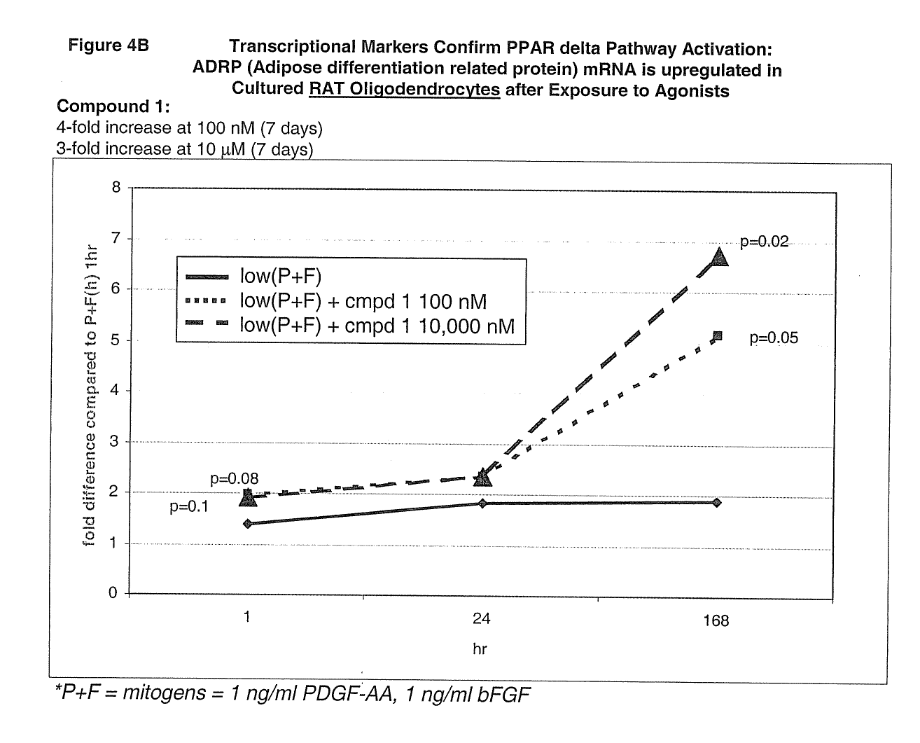

Results for these assays are shown in FIGS. 2, 3, 4A, and 4B. These results suggest that PPAR delta selective agonists bind the PPAR delta receptor and directly activate the PPAR delta pathway in oligodendrocytes and should act similarly in vivo.

Rat Elisa Assay: To evaluate compound induced PPAR delta pathway activation and the extent of oligodendrocyte maturation (changes in protein levels).

1. Plates are washed with PBS, and then keep on ice. Add 200 □l ice old lysis buffer (Tris 50 mM, pH 7.4, MgCI2 2 mM, EDTA 1 mM, β-mercaptoethanol 5 mM, Nonidet P-40 1%, Protease inhibitor cocktail (Roche): 1 tablet/50 ml) to each well.
2. Lyse cells by using pipette to up down and spin plates at 2000 rpm at 4° C. for 5 min. The supernatant is ready to use.
3. Pipet 50 μl of standard, controls and samples to the wells.
4. Add 50 □l of MBP Assay Buffer to each well.
5. Incubate the well, shaking at 500-700 rpm on orbital microplate shaker for 2 hr at room temperature.
6. Add 100 ul of the MBP Antibody-Biotin Conjugate to each well.
7. Incubate the well, shaking at 500-700 rpm on orbital microplate shaker for 1 hr at room temperature.
8. Wash well 5 times with Wash Solution. Blot dry by inverting the plate on absorbent material.
9. Dilute the streptavidin-enzyme conjugate concentrate 1:50 with MBP Elisa Assay buffer. (must be diluted immediately prior to use in the assay).
10. Add 100 μl streptavidin-enzyme conjugate solutions to each well.
11. Incubate the well, shaking at 500-700 rpm on orbital microplate shaker for 30 min at room temperature.
12. Wash well 5 times with the Wash Solution. Blot dry by inverting the plate on absorbent material.
13. Add 100 μl of TMB Chromogen Solution to each well.
14. Incubate the well, shaking at 500-700 rpm on orbital microplate shaker for 10-20 min at room temperature. Avoid exposure to direct sunlight.
15. Add 100 □l of the Stopping Solution to each well.
16. Read the absorbance of the solution in the wells within 30 min, using a microplate reader set to 450 nM.

The above results taken in general and shown in FIGS. 1-6 illustrate that PPAR delta agonists promote oligodendrocyte differentiation even in the presence of mitogens, which normally keep cells mitotically active and inhibit cellular differentiation. Thus, it is expected that in the injured or diseased CNS selective PPAR delta agonists will cause dividing oligodendrocyte progenitor cells to express myelin proteins and ensheath demyelinated or hypomyelinated axons.

### Vivo Proof of Concept Models

Focal Lesions: (used to assess whether compounds protect myelin integrity or accelerate/enhance the rate of remyelination.)

1. Rats 7 weeks of age are given free access to food and water and acclimatized for a minimum of 4 days before use in experiments.
2. Prior to surgery each animal is weighed. The rat is then anaesthetized with ketamine (100 mg/ml) in combination with xylazine (20 mg/ml) in a ratio of 1.8:1. The rats are injected with 0.15 ml/180 g body weight i.p. of the anaesthetic solution prior to the surgical procedure. The animal is prepared for surgery using aseptic conditions in accordance with the IACUC guidelines. All surgical instruments will be autoclaved. The hair is clipped between the ears and this region will then be scrubbed with Betadine, flushed with sterile saline and finally wiped with a pre-packaged sterile alcohol swab.
3. For the surgical procedure, the rat is placed on its ventral surface in a small animal stereotaxic instrument designed to hold the head steady. The incisor bar is always set at −3.9 mm, since this has been shown to achieve a flat-skull position for SD rats.
4. An incision is made in the previously shaven skin overlying the skull between the ears.
5. A small area of bone (0.75 mm in diameter) is drilled at the following coordinates AP −1.8, ML −3.1 from lambda.
6. The bone is removed and rats are injected with 2 μl ethidium bromide, lysolecithin, or SIN-1 into the right caudal cerebellar peduncle, DV −7.1 mm, over a 2 min period by means of a Hamilton μl syringe and needle. Alternatively injections are made into the spinal cord, corpus callosum, or cortex.
7. The needle is left in position for the subsequent 2 min.
8. After withdrawal of the needle the incision is sutured.
9. Each rat receives an i.m. injection of 0.003 mg buprenorphine into a hind leg.
10. The rat is placed in a warming cupboard until it regains consciousness. At which time it is returned to its home cage. Do not allow more than 2 rats per cage, as they will pull each other's suture out.
11. Similar procedures are also done using mice.

Rat Experimental Allergic Encephalomyelitis (Rat EAE) Disease Model:

Experimental allergic encephalomyelitis (EAE) is a T-cell-mediated autoimmune disease of the nervous system that develops in susceptible animals following sensitization with either whole spinal cord homogenate or a component (myelin basic protein). The EAE rodent model is an appropriate tool for studying the inflammation of the brain and spinal cord observed in MS patients. In rodents, injection of whole spinal cord or spinal cord components such as myelin basic protein induces an autoimmune response based on the activation of T-lymphocytes. Clinical disease typically becomes manifest around day 8-10 after inoculation, observed as a broad spectrum of behavioral anomalies ranging from mild gait disturbances and tail atony to complete paralysis and death. Weight loss typically occurs. In animals that survive, spontaneous recovery occurs, accompanied by variable recovery of most motor function. Depending on the species, allergen, and methodology used, animals tested by the EAE model may experience a single (acute EAE) or several (chronic relapsing EAE) attacks. Several treatment paradigms may be used: the drug or treatment of choice may be administered before immunization, during the nonsymptomatic period or during the clinical disease.

Animals:

Female Lewis rats, 160-220 g (Charles River)

Antigen:

Whole Guinea Pig spinal cord (Harlan Biosciences).
Complete Freund's adjuvant H37 Ra [1 mg/ml Mycobacterium Tuberculosis H37 Ra] (Difco).
   Additional Antigen:
Mycobacterium Tuberculosis (Difco).
Bordetella Pertussis [Heat Killed] (Difco).
   Antigen Preparation: (For Approximately 720 Animals):
1. Weigh 5 grams of frozen guinea pig spinal cord.
2. Add 5 g spinal cord to 5 ml 0.9% saline (1 g/ml) in a round bottom centrifuge tube
3. Homogenize on ice with the Tissue-tech until the tissue is completely disrupted (approximately 5 minutes).
4. Add 10 ml Complete Freund's adjuvant H37 Ra supplemented with 200 mg Mycobacterium Tuberculosis (20 mg/ml Complete Freund's adjuvant H37 Ra).
5. Extract homogenate/adjuvant from tube by sucking it into a 10 ml syringe fitted with an 18 gauge emulsifying needle.
6. Emulsify between two 30 ml glass syringes until it becomes difficult to continue passing the material through the needle. (Approximately 5 minutes {there must be no separation between the oil phase and the aqueous phase}).
7. Use immediately or keep on ice until needed (not more than 30 min) (do not freeze).

### Protocol

1. Female Lewis rats (Charles River) are given free access to food and water and should be acclimated a minimum of 3 days before use in experiments.
2. Rats weighing 160 and 220 grams are initially induced with 5% isoflurane (Aerrane, Fort Dodge), 30% O₂, 70% N₂O for 2-5 minutes.
3. The rat is then placed onto a circulating water heating blanket (Gaymar) (dorsal surface up) and into the nose cone for spontaneous respiration of anesthetic gases. The isoflurane is reduced to 2%.
4. Two subcutaneous injections (0.1 ml each) of either antigen or normal saline are made into ventral surface of the hind paws.
5. The animals are removed from the nose cone, weighed and numbered.
6. The rats are allowed to awake from anesthesia and are placed into individual cages.

7. The animals are observed daily for signs of EAE induction (see criteria below)

| | |
|---|---|
| STAGE: 0 | NORMAL |
| STAGE 1 | Abnormal gate and tail atony |
| STAGE 2 | Mild but definite weakness of one or both hind legs |
| STAGE: 3 | Severe weakness of one or both hind legs or mild ataxia |
| STAGE: 4 | Severe paraparesis and minimal hind leg movement |
| STAGE: 5 | No hind leg movement and paraplegia |
| STAGE: 6 | Moribund state with no spontaneous movement and |
| | impaired respiration. Increasing degree of front leg |
| | involvement and urinary and fecal incontinence may |
| | also occur |
| STAGE: 7 | DEATH |

Treatment is begun on day 10 after immunization. Since the disease symptoms in this model typically appear 10-11 days after inoculation, this time point may be considered to represent the initial phase of an acute episode of MS. It is judged that this delay of the start of treatment mimics the clinical situation more closely than the traditionally used protocols where drugs are administered at the time of, or even before, inoculation (Teitelbaum D. et al., Proc Natl Acad Sci USA 1999; 96: 3842-3847 and Brod S. A., et al., Ann Neurol 2000; 47: 127-131).

What we claim is:

1. A method for the treatment of demyelinating diseases in a patient comprising the administration of a therapeutically effective amount of a compound selected from the group comprising one or more compounds of formula (1) and formula (2)

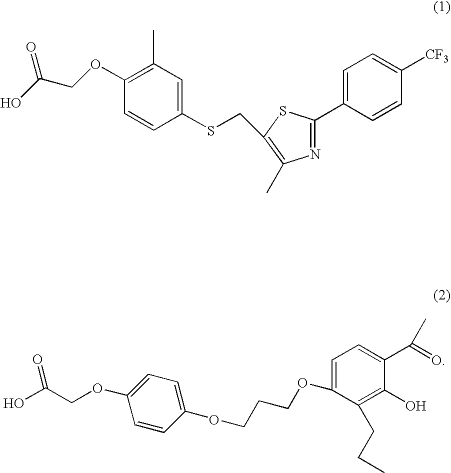

2. The method according to claim 1 wherein said demyelinating disease is selected form the group consisting of multiple sclerosis, Charcot-Marie-Tooth disease, Pelizaeus-Merzbacher disease, encephalomyelitis, neuromyelitis optica, adrenoleukodystrophy, Guillian-Barre syndrome and disorders in which myelin forming glial cells are damaged including spinal cord injuries, neuropathies and nerve injury.

3. The method according to claim 2 wherein the demyelinating disease is multiple sclerosis.

4. A pharmaceutical composition comprising a compound selected from the group consisting of compound of formula (1) and formula (2) in an amount effective for treating multiple sclerosis, Charcot-Marie-Tooth disease, Pelizaeus-Merzbacher disease, encephalomyelitis, neuromyelitis optica, adrenoleukodystrophy, Guillian-Barre syndrome and disorders in which myelin forming glial cells are damaged including spinal cord injuries, neuropathies and nerve injury in combination with at least on pharmaceutically acceptable carrier

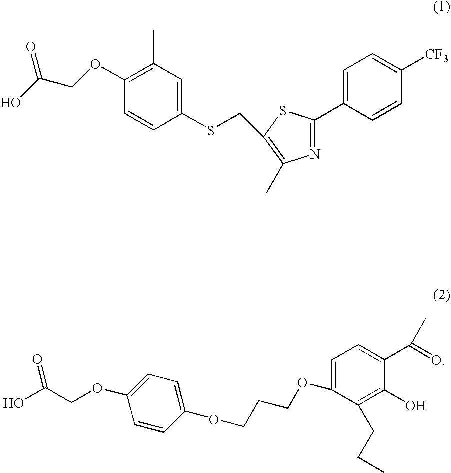

5. The pharmaceutical composition according to claim 4 comprising an amount effective for treating multiple sclerosis.

6. The use of a compound selected from the group consisting of compound of formula (1) and formula (2)

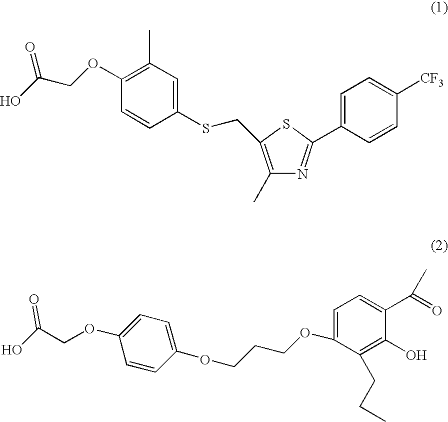

7. A process for the preparation of a pharmaceutical composition for the treatment of a demyelinating disease comprising a mixture of compounds formula (1) and formula (2) together with one or more pharmaceutically acceptable carriers.

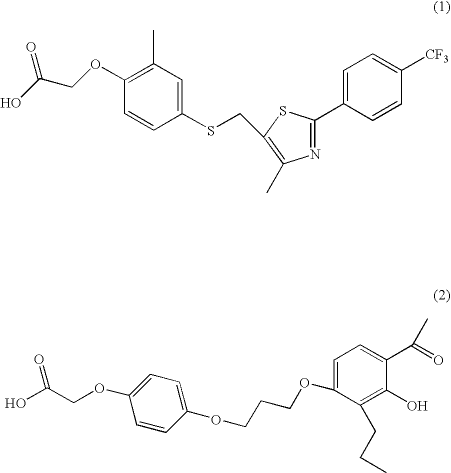

8. The method of claim 6 wherein said demyelinating disease is selected from multiple sclerosis, Charcot-Marie-Tooth disease, Pelizaeus-Merzbacher disease, encephalomyelitis, neuromyelitis optica, adrenoleukodystrophy, Guillian-Barre syndrome and disorders in which myelin forming glial cells are damaged including spinal cord injuries, neuropathies and nerve injury.

9. The method of claim 7 wherein said demyelinating disease is selected from multiple sclerosis, Charcot-Marie-Tooth disease, Pelizaeus-Merzbacher disease, encephalomyelitis, neuromyelitis optica, adrenoleukodystrophy, Guillian-Barre syndrome and disorders in which myelin forming glial cells are damaged including spinal cord injuries, neuropathies and nerve injury.

10. A method for the treatment of demyelinating diseases in a patient comprising the administration of a therapeutically effective amount of a hPPAR delta agonist selected from the group consisting of one or more compounds of formula (1) and formula (2) according to claim 1.

11. The method according to claim 9 wherein the hPPAR delta agonist is a selective agonist.

12. The method according to claim 10 wherein said demyelinating disease is selected from the group comprising multiple sclerosis, Charcot-Marie-Tooth disease, Pelizaeus-Merzbacher disease, encephalomyelitis, neuromyelitis optica, adrenoleukodystrophy, Guillian-Barre syndrome and disorders in which myelin forming glial cells are damaged including spinal cord injuries, neuropathies and nerve injury.
